(19) United States
(12) Patent Application Publication
(10) Pub. No.: US 2002/0165176 A1
(43) Pub. Date: Nov. 7, 2002

(54) Nucleic acid immunization

(76) Inventors: Joel R. Haynes, Madison, WI (US); Michael D. Macklin, Madison, WI (US); Lendon G. Payne, Madison, WI (US)

Correspondence Address: Thomas P. McCracken, POWDERJECT TECHNOLOGIES INC., 6511 Dumbarton Circle, Fremont, CA, 94555, US

(21) Appl. No.: 09/846,091

(22) Filed: Apr. 30, 2001

Related U.S. Application Data
(60) Provisional application No. 60/200,968, filed on May 1, 2000.
(60) Provisional application No. 60/210,580, filed on Jun. 8, 2000.

(51) Int. Cl. A61K 48/00

(52) U.S. Cl. 514/44

(57) ABSTRACT

Recombinant nucleic acid molecules are described. The molecules have a sequence or sequences encoding an influenza virus M2 antigen. Vectors and compositions containing these molecules are also described. Methods for eliciting an immune response using these molecules and compositions are also described.

### CROSS-REFERENCE TO RELATED APPLICATIONS

This application is related to U.S. provisional application serial No. 60/200,968, filed May 1, 2000, and U.S. provisional application serial No. 60/210,580, filed Jun. 8, 2000, from which applications priority is claimed pursuant to 35 U.S.C. §119(e)(1) and which applications are incorporated herein by reference in their entirety.

### TECHNICAL FIELD

The invention relates to the fields of molecular biology and immunology, and generally relates to nucleic acid immunization techniques. More specifically, the invention relates to polynucleotides encoding an influenza antigen, and to nucleic acid immunization strategies employing such polynucleotides.

### BACKGROUND

Techniques for the injection of DNA and mRNA into mammalian tissue for the purposes of immunization against an expression product have been described in the art. The techniques, termed “nucleic acid immunization” herein, have been shown to elicit both humoral and cell-mediated immune responses. For example, sera from mice immunized with a DNA construct encoding the envelope glycoprotein, gp160, were shown to react with recombinant gp160 in immunoassays, and lymphocytes from the injected mice were shown to proliferate in response to recombinant gp120. Wang et al. (1993) Proc. Natl. Acad. Sci. USA 90:4156-4160. Similarly, mice immunized with a human growth hormone (hGH) gene demonstrated an antibody-based immune response. Tang et al. (1992) Nature 356:152-154. Intramuscular injection of DNA encoding influenza nucleoprotein driven by a mammalian promoter has been shown to elicit a CD8+ CTL response that can protect mice against subsequent lethal challenge with virus. Ulmer et al. (1993) Science 259:1745-1749. Immunohistochemical studies of the injection site revealed that the DNA was taken up by myeloblasts, and cytoplasmic production of viral protein could be demonstrated for at least 6 months.

### SUMMARY OF THE INVENTION

It is a primary object of the invention to provide a polynucleotide vaccine composition containing a nucleic acid sequence that encodes at least one influenza virus M2 antigen. The nucleic acid sequence is not present in a recombinant viral vector. The composition can be used as a reagent in various nucleic acid immunization strategies.

It is also a primary object of the invention to provide a method for eliciting an immune response against one or more influenza viruses in an immunized subject. The method entails transfecting cells of the subject with a polynucleotide vaccine composition according to the present invention, that is, a composition containing a sequence that encodes at least one influenza virus M2 antigen. Expression cassettes and/or vectors containing any one of the nucleic acid molecules of the present invention can be used to transfect the cells, and transfection is carried out under conditions that permit expression of the antigens within the subject. The method may further entail one or more steps of administering at least one secondary composition to the subject.

The transfection procedure carried out during the immunization can be conducted either in vivo, or ex vivo (e.g., to obtain transfected cells which are subsequently introduced into the subject prior to carrying out the secondary immunization step). When in vivo transfection is used, the recombinant nucleic acid molecules can be administered to the subject by way of intramuscular or intradermal injection of plasmid DNA or, preferably, administered to the subject using a particle-mediated delivery technique. Secondary vaccine compositions can include the M2 antigen of interest, or other influenza antigens in the form of any suitable vaccine composition, for example, in the form of a peptide subunit composition, in the form of a nucleic acid vaccine composition, or in the form of a whole or split virus influenza vaccine composition.

Advantages of the present invention include, but are not limited to: (i) providing recombinant polynucleotides encoding an influenza virus M2 antigen; and (ii) use of these polynucleotides as reagents in nucleic acid immunization strategies to attain a broadly protective immune response against influenza virus infection.

These and other objects, aspects, embodiments and advantages of the present invention will readily occur to those of ordinary skill in the art in view of the disclosure herein.

### BRIEF DESCRIPTION OF THE DRAWINGS

FIG. 1 depicts an amino acid sequence alignment of the extracellular domains of the M2 proteins of 37 different influenza type A strains, wherein the amino acid residues in bold text denote the variable amino acid positions.

FIG. 2 shows the M2 coding sequence for the influenza strain A/Kagoshima/10/95 (H3N2) that was used in the methods of Example 1.

FIG. 3 is a restriction map and functional map of plasmid pM2-FL that encodes an influenza M2 protein. The M2 coding sequence of pM2-FL was derived using the RNA of influenza virus A/Sydney/5/97 (H3N2) as a template.

FIG. 4 is an annotated depiction of the nucleotide sequence of the pM2-FL plasmid.

FIG. 5 depicts the geometric mean levels of influenza virus in the vaccinated and control animals assessed in the study of Example 5.

### DETAILED DESCRIPTION OF THE INVENTION

Before describing the present invention in detail, it is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise.

Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

The term “nucleic acid immunization” is used herein to refer to the introduction of a nucleic acid molecule encoding one or more selected antigens into a host cell for the in vivo expression of the antigen or antigens. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.”

By “core carrier” is meant a carrier on which a guest nucleic acid (e.g., DNA, RNA) is coated in order to impart a defined particle size as well as a sufficiently high density to achieve the momentum required for cell membrane penetration, such that the guest molecule can be delivered using particle-mediated techniques (see, e.g., U.S. Pat. No. 5,100,792). Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “needleless syringe” is meant an instrument which delivers a particulate composition transdermally without the aid of a conventional needle to pierce the skin. Needleless syringes for use with the present invention are discussed throughout this document.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Thus, the term encompasses delivery from a needleless syringe deliver as described in U.S. Pat. No. 5,630,796, as well as particle-mediated delivery as described in U.S. Pat. No. 5,865,796.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

An “antigen” refers to any agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a protein molecule or portion thereof which contains one or more epitopes. For purposes of the present invention, antigens can be obtained or derived from any appropriate source. Furthermore, for purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens.

By “subunit vaccine” is meant a vaccine composition which includes one or more selected antigens but not all antigens, derived from or homologous to, an antigen from a pathogen of interest such as from a virus, bacterium, parasite or fungus. Such a composition is substantially free of intact pathogen cells or pathogenic particles, or is the lysate of such cells or particles. Thus, a “subunit vaccine” can be prepared from at least partially purified (preferably substantially purified) immunogenic polypeptides from the pathogen, or analogs thereof. Methods for obtaining an antigen to be included in a subunit vaccine can thus include standard purification techniques, recombinant production, or synthetic production. Commercially available influenza subunit vaccines include the FLUVIRIN™ (Evans Medical Limited, Medeva) product which is a purified surface antigen (HA) preparation.

The term “whole virus vaccine” refers to a vaccine composition that contains entire virions that have been inactivated or killed. An example of a whole virus vaccine is the inactivated poliovirus vaccine. A “live attenuated virus vaccine” refers to whole virus vaccine formed with an infectious but weakened virus strain that induces immunity but no disease in a vaccinated subject. Such strains are generally weakened by virus culture in unnatural host cells. Examples of live attenuated virus vaccines include the conventional measles, mumps and rubella vaccines.

By “split vaccine” is meant a vaccine composition that is constituted of virions that have been subjected to treatment with agents such as detergents which dissolve lipids to disrupt the virions, allowing the removal of pyrogenic substances. The term can be used interchangeably with the terms “split virus,” “split virion” and “split antigen” which have the same meaning herein. Commercially available influenza split vaccines include the FLU-SHIELD™ (Wyeth-Lederle Laboratories) product, the FLUZONE™ (Pasteur-Merieux Connaught Laboratories) product and the FLUOGEN™ (Parkdale) product.

An “immune response” against an antigen of interest is the development in an individual of a humoral and/or a cellular-immune response to that antigen. For purposes of the present invention, a “humoral immune response” refers to an immune response mediated by antibody molecules, while a “cellular immune response” is one mediated by T-lymphocytes and/or other white blood cells.

The terms “nucleic acid molecule” and “polynucleotide” are used interchangeably herein and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term nucleic acid sequence is the alphabetical representation of a polynucleotide molecule. This alphabetical representation can be input into databases in a computer having a central processing unit and used for bioinformatics applications such as functional genomics and homology searching.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors. A “plasmid” is a vector in the form of an extrachromosomal genetic element.

A nucleic acid sequence which “encodes” a selected antigen is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, procaryotic or eucaryotic mRNA, genomic sequences from viral or procaryotic DNA or RNA, and even synthetic DNA sequences. A transcription termination sequence may be located 3′ to the coding sequence.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

“Recombinant” is used herein to describe a nucleic acid molecule (polynucleotide) of genomic, cDNA, semisynthetic, or synthetic origin which, by virtue of its origin or manipulation is not associated with all or a portion of the polynucleotide with which it is associated in nature and/or is linked to a polynucleotide other than that to which it is linked in nature. Two nucleic acid sequences which are contained within a single recombinant nucleic acid molecule are “heterologous” relative to each other when they are not normally associated with each other in nature.

Techniques for determining nucleic acid and amino acid “sequence identity” or “sequence homology” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5×Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

The term “adjuvant” intends any material or composition capable of specifically or non-specifically altering, enhancing, directing, redirecting, potentiating or initiating an antigen-specific immune response. Thus, coadministration of an adjuvant with an antigen may result in a lower dose or fewer doses of antigen being necessary to achieve a desired immune response in the subject to which the antigen is administered, or coadministration may result in a qualitatively and/or quantitatively different immune response in the subject. The effectiveness of an adjuvant can be determined by administering the adjuvant with a vaccine composition in parallel with vaccine composition alone to animals and comparing antibody and/or cellular-mediated immunity in the two groups using standard assays such as radioimmunoassay, ELISAs, CTL assays, and the like, all well known in the art. Typically, in a vaccine composition, the adjuvant is a separate moiety from the antigen, although a single molecule can have both adjuvant and antigen properties (e.g., cholera toxin).

An “adjuvant composition” intends any pharmaceutical composition containing an adjuvant. Adjuvant compositions can be delivered in the methods of the invention while in any suitable pharmaceutical form, for example, as a liquid, powder, cream, lotion, emulsion, gel or the like. However, preferred adjuvant compositions will be in particulate form. It is intended, although not always explicitly stated, that molecules having similar biological activity as wild-type or purified peptide or chemical adjuvants, and nucleic acid encoding adjuvant molecules can be used within the spirit and scope of the invention.

The terms “individual” and “subject” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

General Overview

The present invention provides novel nucleic acid molecules containing a sequence that encodes an influenza virus M2 antigen. The M2 antigen sequence is not present in a recombinant viral vector. These molecules are useful in eliciting an immune response in a subject against influenza virus. In particular, the present inventors have determined that, surprisingly, a nucleic acid immunization technique (e.g, particle-mediated delivery of core carrier particles coated with the nucleic acid molecules of the present invention) can be used to elicit an immune response against influenza virus in an immunized subject, and that the resultant immune response provides protection against disease associated with infection of essentially all influenza virus type A strains due to the exceptionally high degree of sequence conservation in the M2 protein among these different strains.

Influenza viruses type A and type B are members of the family of Orthomyxoviruses and are important human pathogens. In particular, influenza, an acute illness caused by infection of the respiratory tract with influenza A or B virus, causes significant morbidity and mortality worldwide due to regular outbreaks occurring nearly every year, necessitating annual vaccination in at-risk population groups such as the elderly and immunocompromised individuals. Influenza also has the potential to be catastrophic due to periodic epidemics and pandemics caused by newly emerging influenza virus strains. These new strains emerge due to the phenomena of antigenic drift and antigenic shift in the influenza virus, which allows these viruses to change or mutate their antigenic make-up. Antigenic drift and shift events result in new strains that can avoid pre-existing immune responses in individuals established by prior vaccination or prior infection. Annual vaccination is thus required for these viruses.

Influenza type A and type B viruses are negative stranded RNA viruses in which the viral genomes are divided into 8 separate single stranded RNA segments. Influenza A and B viruses represent different members of the same genus and are morphologically indistinguishable, but share no serological cross-reactivity. In addition, the type A and type B viruses are different enough that individual RNA segments from one virus cannot be substituted for the homologous or similar segment of the other virus.

The nucleocapsid core of influenza type A and B viruses contains the 8 RNA segments and is found within a lipid envelope (of cellular origin) along with several internal structural and nonstructural proteins that include nucleoprotein (NP), matrix (M1), polymerase (PB1, PB2, and PA), and nonstructural (NS1 and NS2) molecules. Integrated into the membrane and exposed to the outer surface of the virus are two major glycoproteins, the hemagglutinin (HA) and neuraminidase (NA) glycoproteins. In addition, a third minor membrane protein, M2, is found in small quantities (20 to 60 molecules per virion).

New influenza virus strains arise by point mutation of individual genes and by reassortment of the 8 genomic RNA segments when an individual or animal is infected simultaneously with more than one viral strain. Point mutations that result in amino acid sequence changes in the HA or NA genes are responsible for the phenomenon of “antigenic drift.” Antigenic drift occurs in both type A and type B viruses. The phenomenon of reassortment is responsible for “antigenic shift” that occurs when an influenza type A virus acquires a new HA or NA gene by virtue virtue of having acquired a new RNA segment. Antigenic shift is limited to type A viruses since type A viruses have multiple serotypes circulating for both the HA and NA genes. Multiple serotypes of the HA and NA genes of the type B viruses do not exist.

The HA and NA antigens (the major surface glycoproteins) are the important antigens in terms of protective immunity against influenza virus infection. Neutralizing antibody responses are directed predominantly against the HA gene product and, to a lesser extent, the NA gene product. A substantial cytotoxic T lymphocyte (CTL) response is also directed against the HA gene product, but this reponse likely plays a limited role in providing protective immunity against disease. The NP, PB1, PB2, PA, M1, M2 and NS1 and NS2 proteins of influenza viruses are markedly more conserved between strains and mutations in these genes do not play a role in the phenomena of antigenic drift and antigenic shift.

Current licensed influenza vaccine products are derived from influenza virus grown in eggs which is then inactivated to provide whole virus vaccine compositions, or further processed to provide split virus vaccine compositions or purified surface antigen vaccine compositions. All of these vaccine products target the HA and/or NA antigens as these are considered to be the most important targets for the induction of antibody responses via vaccination. While antibody and cellular immune responses arise against other influenza antigens, it has been amply demonstrated that protective immunity is mediated predominantly by serum and mucosal antibody responses specific for the HA and NA antigens. Animal models support this dogma in which it has been shown that passive transfer of monoclonal antibodies to HA or NA antigens is protective against challenge, whereas passive transfer of antibodies specific for NP or M antigens provides little or no protection at all. Askonas et al. (1982) “The immune response to influenza viruses and the problem of protection against infection,” in Basic and applied influenza research, Beare A. S. ed, CRC Press, Boca Raton Fla., pp159-188.

In contrast to HA and NA, the influenza M2 antigen has generally been ignored as a vaccine candidate due to the limiting quantities of M2 on influenza virions and the fact that M2-specific antibodies that arise in patients following infection are weak, transient and sporadic. Black et al. (1993) J. Gen. Virol. 74:143-146. However, unlike the HA and NA antigens, the M2 protein is very highly conserved throughout all influenza type A isolates, independent of subtype. The high degree of conservation of M2 among all type A isolates indicates that this protein is likely not an important antigen in terms of the host's natural reaction to an influenza virus infection. If natural M2-specific responses were responsible for eliciting protection against infection, there would likely be selective pressure for considerable heterogeneity in the M2 amino acid sequence such as there is for HA and NA.

Even though M2-specific antibody responses are not abundantly induced following a natural infection, recent attempts to produce alternative influenza vaccines have used M2 as a potential vaccine antigen, where recombinant M2 proteins, or M2 subfragments, have been produced in recombinant expression systems and used as candidate subunit vaccines (see, e.g., International Publication Nos. WO 93/03173, published Feb. 18, 1993, and WO 99/28478, published Jun. 10, 1999, and US Pat. No. 5,691,189 to Kurtz et al.). The basic assumption behind the use of recombinant M2 proteins in these vaccine compositions is that they should induce a more vigorous and durable M2-specific response than otherwise elicited in a natural infection. This effect has in fact been reported by several groups which produced the M2 protein (or parts thereof) in recombinant expression systems and then used the same as a recombinant subunit vaccine in mice (Slepushkin et al. (1995) Vaccine 13:1399-1402; Frace et al. (1999) Vaccine 17:2237-2244; and Neirynck et al. (1999) Nature Med. 5:1157-1163). In all three cases, M2-specific antibody responses elicited by vaccination were capable of protecting mice from death (but not infection) in a lethal mouse influenza challenge model.

In light of the relative successes seen with recombinant M2 vaccine compositions, it may seem that the use of a live virus vector to supply M2 antigen in vivo would be an ideal means of producing a vigorous and durable M2-specific antibody response. However, in stark contrast to such expectations, past attempts at immunization by inserting a DNA sequence encoding the M2 protein in vaccinia virus recombinant vectors and immunizing mice and ferrets with such vectors have failed, resulting in no measurable vaccine protection (Epstein et al. (1993) J. Immunol. 150:5484-5493 and Jakeman et al. (1989) J. Gen. Virol. 70:1523-1531). From this observation, it seems that M2 expression induced in vivo (e.g., by natural influenza virus infection or by nucleic acid immunization techniques) is not effective in inducing protective M2-specific antibody responses. In contrast, the formulation of M2 recombinant subunit proteins, protein fragments, or fusion proteins seems better suited for the elicitation of M2-specific antibody responses that will afford protection from symptoms associated with influenza virus infection.

As noted above, the present invention relates to the surprising discovery that a nucleic acid immunization technique can be used to provide a robust, M2-specific immune response, and that this immune response is able to provide vaccine protection against influenza disease. Thus, in one embodiment of the invention, a polynucleotide vaccine composition is provided, wherein the composition contains a nucleic acid sequence encoding an influenza virus M2 antigen. The nucleic acid sequence is not present in a recombinant viral vector. The M2 antigen is obtained or derived from the M2 protein of influenza type A virus. The M2 protein of influenza type A virus is a small, 97 amino acid integral membrane protein. M2 is found in limited quantities in the virion (Zebedee et al. (1988) J. Virol. 62:2762-2772), but in much larger quantities integrated into the membrane of an infected cell (Lamb et al. (1985) Cell 40:627-633). M2 is a type III integral membrane protein in that its N-terminus is exposed to the outside of the cell, but lacks a cleavable signal peptide sequence. Adjacent to the 24 amino acid N-terminal extracellular domain is a 19 amino acid transmembrane domain, followed by a 54 amino acid cytoplasmic tail at the C-terminus. The M2 protein exists in the membrane of infected cells and virions as a homotetramer composed of two disulfide linked dimers that are attached noncovalently. The M2 homotetramer has ion channel activity that is required for completion of the influenza infectious cycle.

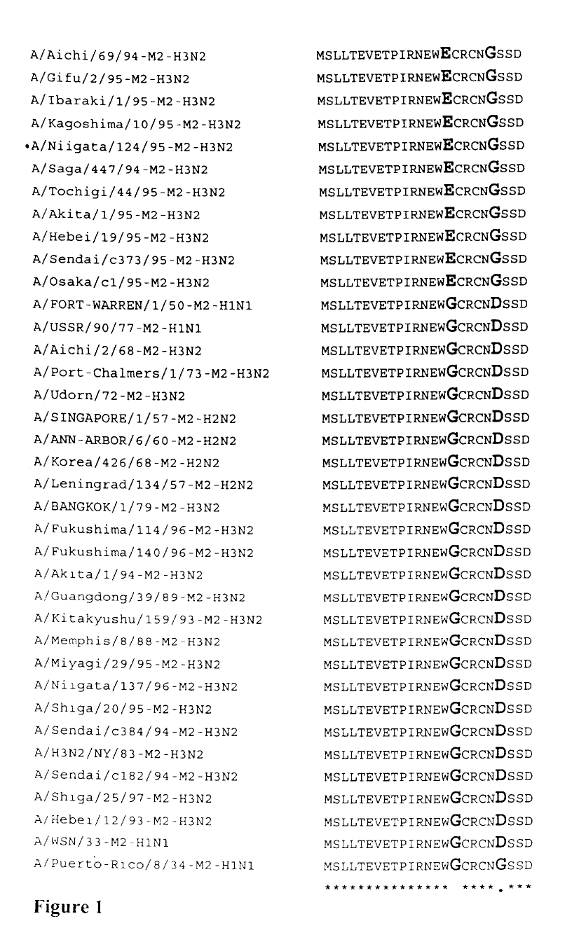

FIG. 1 shows a sequence comparison of the 24 amino acid extracellular domain of the M2 protein from 37 different influenza type A isolates. These 37 isolates represent a span of 61 years of evolutionary history of the influenza A and include three different subtypes (H3N2, H2N2, and H1N1). As noted in the figure, there are only two variable amino acid positions in the extracellular domain among the 37 isolates (amino acid positions 16 and 21). Moreover, each of these two positions has only two possible amino acid residues and only three combinations of the two variable positions are observed, resulting in only three extracellular domains: MSLLTEVETPIRNEWECRCNGSSD (SEQ ID NO:1); MSLLTEVETPIRNEWGCRCNDSSD (SEQ ID NO:2); and MSLLTEVETPIRNEWGCRCNGSSD (SEQ ID NO:3). Thus, in certain embodiments, the present polynucleotide vaccine compositions contain a nucleic acid sequence encoding an influenza virus M2 polypeptide that comprises the amino acid sequence of one or more of SEQ ID NO:1, SEQ ID NO:2, SEQ ID NO:3, or hybrids or combinations thereof. In one particular embodiment, a hybrid sequence is used where the first variable amino acid position was derived from the M2 sequence of influenza virus A/Kagoshima/10/95 and the second variable amino acid position was derived from the M2 sequence of influenza virus A/Sydney. This particular M2-encoding sequence is present in the vector pM2-FL described in detail herein below. The high degree of conservation within M2 from various isolates suggests that the vaccine strategies of the present invention have the potential to elicit antibody responses that are broadly protective.

The polynucleotide vaccine compositions of the invention can be used as standalone vaccines, or as part of a multi-component vaccine composition. For example, in a multi-component vaccine composition, the present nucleic acid molecules are combined with additional nucleic acid molecules encoding additional influenza antigens known to be important for providing protection against influenza, for example, molecules containing sequences that encode influenza HA or NA antigens. Alternatively, the multi-component vaccine composition may contain conventional whole virus, split virus or purified viral subunit influenza vaccine preparations that are rich in the HA antigen. These additional components help provide immune responses that are more strain-specific due to the variability of the HA and NA antigens from strain to strain, and from year to year. The M2 nucleic acid vaccine component of these multi-component vaccine compositions helps complement the efficacy of these more traditionally based influenza vaccine compositions by allowing for broadly protective rather than strain-specific immune responses in vaccinated subjects. Thus, the invention provides more effective vaccines and methods of immunization against infection with influenza virus.

Polynucleotides

In one embodiment, a recombinant polynucleotide vaccine composition is provided. The composition includes one or more nucleic acid molecules that contain a sequence encoding an influenza virus M2 antigen. In one particular embodiment, a cocktail of nucleic acid molecules is provided, where at least one nucleic acid molecule in the cocktail has a sequence encoding a M2 antigen.

The entire genomes of the influenza type A and B viruses have been sequenced and the sequences are publically available, for example on the World Wide Web, and are deposited with GENBANK. In particular, the DNA sequences of the M2 genes of numerous influenza A viruses are known and are readily available (Ito et al. (1991) J. Virol. 65:5491-5498). Active variants of these antigen sequences may also be used in the compositions and methods of the present invention. Sequences encoding the selected M2 antigen are typically inserted into an appropriate vector (e.g., a plasmid backbone) using known techniques and as described below in the Examples.

The sequence or sequences encoding the influenza M2 antigen of interest can be obtained and/or prepared using known methods. For example, substantially pure antigen preparations can be obtained using standard molecular biological tools. That is, polynucleotide sequences coding for the above-described antigens can be obtained using recombinant methods, such as by screening CDNA libraries from cells expressing an antigen, or by deriving the coding sequence for the M2 antigen from a vector known to include the same. The M2 sequence can also be obtained directly from the RNA of purified type A influenza virus. Many influenza virus strains are on deposit with the American Type Culture Collection ATCC, and yet others are available from national and international health organizations such as the Centers of Disease Control (Atlanta, Ga.). See, e.g., Sambrook et al., supra, for a description of techniques used to obtain and isolate nucleic acid molecules. Polynucleotide sequences can also be produced synthetically, rather than cloned.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used.

These same techniques can be used to obtain sequences encoding other influenza antigens. The relative ease of producing and purifying nucleic acid constructs facilitates the generation of combination vaccines, for example, polynucleotide vaccine compositions that contain one or more nucleic acid molecules containing an M2 sequence in combination with other M2 sequences or further influenza antigen sequences (sequences encoding NP, HA, NA, M1, PB1, PB2, PA, NS1 and/or NS2 antigens).

Once the relevant sequences for the M2 antigens of interest and, alternatively, sequences encoding other influenza antigens such as HA or NA antigens, have been obtained, they can be linked together to provide one or more contiguous nucleic acid molecules using standard cloning or molecular biology techniques. More particularly, after sequence information for one or more M2 antigens of interest have been obtained, they can be combined with each other or with other sequences to form a hybrid sequence, or handled separately. In hybrid sequences, the various antigen sequences can be positioned in any manner relative to each other, and be included in a single molecule in any number ways, for example, as a single copy, randomly repeated in the molecule as multiple copies, or included in the molecule as multiple tandem repeats or otherwise ordered repeat motifs.

Although any number of routine molecular biology techniques can be used to construct such recombinant nucleic acid molecules, one convenient method entails using one or more unique restriction sites in a shuttle or cloning vector (or inserting one or more unique restriction sites into a suitable vector sequence) and standard cloning techniques to direct the influenza virus M2 antigen sequence or sequences to particular target locations within a vector.

Alternatively, hybrid molecules can be produced synthetically rather than cloned. The nucleotide sequence can be designed with the appropriate codons for the particular amino acid sequence desired. In general, one will select preferred codons for the intended host in which the sequence will be expressed. The complete sequence can then be assembled from overlapping oligonucleotides prepared by standard methods and assembled into a complete coding sequence. See, e.g., Edge (1981) Nature 292:756; Nambair et al. (1984) Science (1984) 223:1299; Jay et al. (1984) J. Biol. Chem. 259:6311.

Once the relevant M2 antigen sequences (and, optionally additional sequences that encode other influenza antigens) have been obtained or constructed, they can be inserted into a vector which includes control sequences operably linked to the inserted sequence or sequences, thus providing expression cassettes that allow for expression of antigen in vivo in a targeted subject species.

Typical promoters for mammalian cell expression include the SV40 early promoter, a CMV promoter such as the CMV immediate early promoter, the mouse mammary tumor virus LTR promoter, the adenovirus major late promoter (Ad MLP), and other suitably efficient promoter systems. Nonviral promoters, such as a promoter derived from the murine metallothionein gene, may also be used for mammalian expression. Inducible, repressible or otherwise controllable promoters may also be used. Typically, transcription termination and polyadenylation sequences will also be present, located 3′ to each translation stop codon. Preferably, a sequence for optimization of initiation of translation, located 5′ to each coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the expression cassette.

In addition, enhancer elements may be included within the expression cassettes in order to increase expression levels. Examples of suitable enhancers include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777), and elements derived from human or murine CMV (Boshart et al. (1985) Cell 41:521), for example, elements included in the CMV intron A sequence.

Adjuvants

Although not required, the polynucleotide vaccine compositions of the present invention may effectively be used with any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); adjuvants formed from bacterial cell wall components such as adjuvants including lipopolysaccharides (e.g., lipid A or monophosphoryl lipid A (MPL), Imoto et al. (1985)Tet. Lett. 26:1545-1548), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM₁₉₇, a non-toxic diphtheria toxin mutant (see, e.g., Bixler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemokines and cytokines, such as interleukins (e.g., IL-1, IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-^{L}-alanyl-^{D}-isoglutamine (nor-MDP), N-acetylmuramyl-^{L}-alanyl-^{D}-isoglutaminyl-^{L}-alanine-2-(1′-2′-dipalmitoyl-sn-glycero-3 huydroxyphosphoryloxy)-ethylamine (MTP-PE) etc.; adjuvants derived from the CpG family of molecules, CpG dinucleotides and synthetic oligonucleotides which comprise CpG motifs (see, e.g., Krieg et al. Nature (1995) 374:546, Medzhitov et al. (1997) Curr. Opin. Immunol. 9:4-9, and Davis et al. J. Immunol (1998) 160:870-876) such as TCCATGACGTTCCTGATGCT (SEQ ID NO:4) and ATCGACTCTCGAGCGTTCTC (SEQ ID NO:5); and synthetic adjuvants such as PCPP (Poly[di(carboxylatophenoxy)phosphazene) (Payne et al. Vaccines (1998) 16:92-98). Such adjuvants are commercially available from a number of distributors such as Accurate Chemicals; Ribi Immunechemicals, Hamilton, Mont.; GIBCO; Sigma, St. Louis, Mo. Preferred adjuvants are those derived from ADP-ribosylating bacterial toxins, with cholera toxin and heat labile toxins being most preferred. Oligonucleotides containing a CpG motif are also preferred. Other preferred adjuvants are those provided in nucleic acid form, for example nucleic acid sequences that encode chemokines and cytokines, such as interleukins (e.g., IL-1, IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2 molecules.

The adjuvant may delivered individually or delivered in a combination of two or more adjuvants. In this regard, combined adjuvants may have an additive or a synergistic effect in promoting a desired immune response. A synergistic effect is one where the result achieved by combining two or more adjuvants is greater than one would expect than by merely adding the result achieved with each adjuvant when administered individually. A preferred adjuvant combination is an adjuvant derived from an ADP-ribosylating bacterial toxin and a synthetic oligonucleotide comprising a CpG motif. A particularly preferred combination comprises cholera toxin and the oligonucleotide

ATCGACTCTCGAGCGTTCTC (SEQ ID NO:5).

Unfortunately, a majority of the above-referenced adjuvants are known to be highly toxic, and are thus generally considered too toxic for human use. It is for this reason that the only adjuvant currently approved for human usage is alum, an aluminum salt composition. Nevertheless, a number of the above adjuvants are commonly used in animals and thus suitable for numerous intended subjects, and several are undergoing preclinical and clinical studies for human use. However, as discussed herein above, the adjuvants are preferably rendered into particulate form for transdermal delivery using a powder injection method. Surprisingly, it has been found that adjuvants which are generally considered too toxic for human use may be rendered into particulate form and administered with a powder injection technique without concomitant toxicity problems. Without being bound by a particular theory, it appears that delivery of adjuvants to the skin, using transdermal delivery methods (powder injection), allows interaction with Langerhans cells in the epidermal layer and dendritic cells in the cutaneous layer of the skin. These cells are important in initiation and maintenance of an immune response. Thus, an enhanced adjuvant effect can be obtained by targeting delivery to or near such cells. Moreover, transdermal delivery of adjuvants in the practice of the invention may avoid toxicity problems because (1) the top layers of the skin are poorly vascularized, thus the amount of adjuvant entering the systemic circulation is reduced which reduces the toxic effect; (2) skin cells are constantly being sloughed, therefore residual adjuvant is eliminated rather than absorbed; and (3) substantially less adjuvant can be administered to produce a suitable adjuvant effect (as compared with adjuvant that is delivered using conventional techniques such as intramuscular injection).

Once selected, one or more adjuvant can be provided in a suitable pharmaceutical form for parenteral delivery, the preparation of which forms are well within the general skill of the art. See, e.g., Remington's Pharmaceutical Sciences (1990) Mack Publishing Company, Easton, Pa., 18th edition. Alternatively, the adjuvant can be rendered into particulate form as described in detail below. The adjuvant(s) will be present in the pharmaceutical form in an amount sufficient to bring about the desired effect, that is, either to enhance the response against the coadministered antigen of interest, and/or to direct an immune response against the antigen of interest. Generally about 0.1 μg to 1000 μg of adjuvant, more preferably about 1 μg to 500 μg of adjuvant, and more preferably about 5 μg to 300 μg of adjuvant will be effective to enhance an immune response of a given antigen. Thus, for example, for CpG, doses in the range of about 0.5 to 50 μg, preferably about 1 to 25 μg, and more preferably about 5 to 20 μg, will find use with the present methods. For cholera toxin, a dose in the range of about 0.1 μg to 50 μg, preferably about 1 μg to 25 μg, and more preferably about 5 μg to 15 μg will find use herein. Similarly, for alum or PCPP, a dose in the range of about 2.5 μg to 500 μg, preferably about 25 to 250 μg, and more preferably about 50 to 150 μg, will find use herein. For MPL, a dose in the range of about 1 to 250 μg, preferably about 20 to 150 μg, and more preferably about 40 to 75 μg, will find use with the present methods.

Doses for other adjuvants can readily be determined by one of skill in the art using routine methods. The amount to administer will depend on a number of factors including the nature of the M2 antigen.

Administration of Polynucleotides

Once complete, the polynucleotide constructs are used for nucleic acid immunization using standard gene delivery protocols. Methods for gene delivery are known in the art. See, further below. The nucleic acid molecules of the present invention can thus be delivered either directly to a subject or, alternatively, delivered ex vivo to cells derived from the subject whereafter the cells are reimplanted in the subject.

Conventional Pharmaceutical Preparations

Formulation of a preparation comprising the above-described recombinant polynucleotide vaccine compositions, with or without addition of an adjuvant composition, can be carried out using standard pharmaceutical formulation chemistries and methodologies all of which are readily available to the ordinarily skilled artisan. For example, compositions containing one or more nucleic acid sequences (e.g., present in a suitable vector form such as a DNA plasmid) can be combined with one or more pharmaceutically acceptable excipients or vehicles to provide a liquid preparation.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the vaccine composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.,J. 1991), incorporated herein by reference.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publicaiton No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated vaccine compositions will include a polynucleotide containing a sequence that encodes the selected M2 antigen or antigens of interest in an amount sufficient to mount an immunological response. An appropriate effective amount can be readily determined by one of skill in the art. Such an amount will fall in a relatively broad range that can be determined through routine trials. For example, immune responses have been obtained using as little as 1 μg of DNA, while in other administrations, up to 2 mg of DNA has been used. It is generally expected that an effective dose of the polynucleotide will fall within a range of about 10 μg to 1000 μg, however, doses above and below this range may also be found effective. The compositions may thus contain from about 0.1% to about 99.9% of the polynucleotide molecules and can be administered directly to the subject or, alternatively, delivered ex vivo, to cells derived from the subject, using methods known to those skilled in the art

Administration of Conventional Preparations

Once suitably formulated, these vaccine compositions can be administered to a subject in vivo using a variety of known routes and techniques. For example, the liquid preparations can be provided as an injectable solution, suspension or emulsion and administered via parenteral, subcutaneous, intradermal, intramuscular, intravenous injection using a conventional needle and syringe, or using a liquid jet injection system. Liquid preparations can also be administered topically to skin or mucosal tissue, or provided as a finely divided spray suitable for respiratory or pulmonary administration. Other modes of administration include oral administration, suppositories, and active or passive transdermal delivery techniques.

Alternatively, the vaccine compositions can be administered ex vivo, for example delivery and reimplantation of transformed cells into a subject are known (e.g., dextran-mediated transfection, calcium phosphate precipitation, electroporation, and direct microinjection of into nuclei).

Coated Particle Pharmaceutical Preparations

In one preferred embodiment, the polynucleotide vaccine compositions (e.g., a DNA vaccine), whether or not combined with conventional influenza vaccine compositions and/or adjuvants are delivered using carrier particles. Particle-mediated methods for delivering such nucleic acid preparations are known in the art. Thus, once prepared and suitably purified, the above-described nucleic acid molecules and/or adjuvants can be coated onto carrier particles (e.g., core carriers) using a variety of techniques known in the art. Carrier particles are selected from materials which have a suitable density in the range of particle sizes typically used for intracellular delivery from a particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

For the purposes of the invention, tungsten, gold, platinum and iridium carrier particles can be used. Tungsten and gold particles are preferred. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (e.g., gold powder A1570, available from Engelhard Corp., East Newark, N.,J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity. Microcrystalline gold provides a diverse particle size distribution, typically in the range of 0.1-5 μm. However, the irregular surface area of microcrystalline gold provides for highly efficient coating with nucleic acids.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular particle-mediated delivery instruments.

Peptides (e.g., an influenza purified subunit vaccine), can also be coated onto suitable carrier particles, e.g., gold or tungsten. For example, peptides can be attached to the carrier particle by simply mixing the two components in an empirically determined ratio, by ammonium sulfate precipitation or solvent precipitation methods familiar to those skilled in the art, or by chemical coupling of the peptide to the carrier particle. The coupling of L-cysteine residues to gold has been previously described (Brown et al., Chemical Society Reviews 9:271-311 (1980)). Other methods include, for example, dissolving the peptide antigen in absolute ethanol, water, or an alcohol/water mixture, adding the solution to a quantity of carrier particles, and then drying the mixture under a stream of air or nitrogen gas while vortexing. Alternatively, the peptide antigens can be dried onto carrier particles by centrifugation under vacuum. Once dried, the coated particles can be resuspended in a suitable solvent (e.g., ethyl acetate or acetone), and triturated (e.g., by sonication) to provide a substantially uniform suspension.

Administration of Coated Particles

Following their formation, carrier particles coated with the nucleic acid preparations and, alternatively, adjuvants and/or peptide, protein, or whole or split virus preparations, can be delivered to a subject using particle-mediated delivery techniques.

Various particle acceleration devices suitable for particle-mediated delivery are known in the art, and are all suited for use in the practice of the invention. Current device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

If desired, these particle acceleration devices can be provided in a preloaded condition containing a suitable dosage of the coated carrier particles comprising the polynucleotide vaccine composition, with or without additional influenza vaccine compositions and/or a selected adjuvant component. The loaded syringe can be packaged in a hermetically sealed container.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 1000 μg, more preferably 0.01 to 10.0 μg of nucleic acid molecule per dose, and in the case of peptide or protein molecules is 1 ug to 5 μg, more preferably 1 to 50 μg of peptide, depends on the subject to be treated. The exact amount necessary will vary depending on the age and general condition of the individual being immunized and the particular nucleotide sequence or peptide selected, as well as other factors. An appropriate effective amount can be readily determined by one of skill in the art upon reading the instant specification.

Particulate Pharmaceutical Preparations

Alternatively, the polynucleotides of the present invention (as well as one or more selected adjuvant and/or conventional influenza vaccine compositions) can also be formulated as a particulate composition. More particularly, formulation of particles comprising the antigen and/or adjuvant of interest can be carried out using the above-described standard pharmaceutical formulation chemistries. For example, the polynucleotides and/or adjuvants can be combined with one or more pharmaceutically acceptable excipient or vehicle to provide a suitable vaccine composition.

The formulated compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in commonly owned International Publication No. WO 97/48485, incorporated herein by reference.

These methods can be used to obtain nucleic acid particles having a size ranging from about 0.01 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Similarly, particles of selected adjuvants having a size ranging from about 0.1 to about 250 μm, preferably about 0.1 to about 150 μm, and most preferably about 20 to about 60 μm; a particle density ranging from about 0.1 to about 25 g/cm³, and a bulk density of preferably about 0.5 to about 3.0 g/cm³, and most preferably about 0.8 to about 1.5 g/cm³ can be obtained.

Single unit dosages or multidose containers, in which the particles may be packaged prior to use, can comprise a hermetically sealed container enclosing a suitable amount of the particles comprising the antigen of interest and/or the selected adjuvant (e.g., the vaccine composition). The particulate compositions can be packaged as a sterile formulation, and the hermetically sealed container can thus be designed to preserve sterility of the formulation until use in the methods of the invention. If desired, the containers can be adapted for direct use in a needleless syringe system. Such containers can take the form of capsules, foil pouches, sachets, cassettes, and the like. Appropriate needleless syringes are described herein above.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

Administration of Particulate Compositions

Following their formation, the particulate composition (e.g., powder) can be delivered transdermally to the subject's tissue using a suitable transdermal delivery technique. Various particle acceleration devices suitable for transdermal delivery of the substance of interest are known in the art, and will find use in the practice of the invention. A particularly preferred transdermal delivery system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect® needleless syringe device”). Other needleless syringe configurations are known in the art and are described herein.

The particulate compositions (comprising the antigen of interest and/or a selected adjuvant) can be administered using a transdermal delivery technique. Preferably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labeled as described above.

Compositions containing a therapeutically effective amount of the powdered molecules described herein can be delivered to any suitable target tissue via the above-described needleless syringes. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid molecule per dose, depends on the subject to be treated. Doses for other pharmaceuticals, such as physiological active peptides and proteins, generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

Thus, a “therapeutically effective amount” of the present particulate compositions will be sufficient to bring about treatment or prevention of disease or condition symptoms, and will fall in a relatively broad range that can be determined through routine trials.

Eliciting Immune Responses

In another embodiment of the invention, a method for eliciting an immune response against an influenza virus in a subject is provided. In essence, the method entails providing a polynucleotide vaccine composition, where the compositions contains a nucleic acid molecule encoding an influenza virus M2 antigen. The nucleic acid molecule is not present in a recombinant viral vector. The nucleic acid sequence encoding the M2 antigen is linked to regulatory sequences to provide an expression cassette. This expression cassette is then provided in a suitable vector, for example a plasmid vector construct. In particular embodiments, the M2 antigen is an influenza virus M2 polypeptide that preferably contains an extracellular domain portion substantially homologous to the 24mer amino acid sequence of SEQ ID NO:1, SEQ ID NO:2, SEQ ID NO:3, or hybrids and combinations thereof.

In one aspect, the method entails administering the vaccine composition to the subject using standard gene delivery techniques that are known in the art. See, e.g., U.S. Pat. Nos. 5,399,346, 5,580,859, 5,589,466. Typically, the polynucleotide vaccine composition is combined with a pharmaceutically acceptable excipient or vehicle to provide a liquid preparation (as described herein above) and then used as an injectable solution, suspension or emulsion for administration via parenteral, subcutaneous, intradermal, intramuscular, intravenous injection using a conventional needle and syringe, or using a liquid jet injection system. It is preferred that the composition be administered to skin or mucosal tissue of the subject. Liquid preparations can also be administered topically to skin or mucosal tissue, or provided as a finely divided spray suitable for respiratory or pulmonary administration. Other modes of administration include oral administration, suppositories, and active or passive transdermal delivery techniques. The polynucleotide vaccine compositions can alternatively be delivered ex vivo to cells derived from the subject, whereafter the cells are reimplanted in the subject. Upon introduction into the subject, the nucleic acid sequence is expressed to provide M2 antigen in situ in an amount sufficient to elicit an anti-influenza immune response in the vaccinated subject. This immune response can be a humoral (antibody) response, a cellular (CTL) response, or be characterized as raising both a humoral and a cellular immune response against the influenza antigen.

It is preferred, however, that the polynucleotide vaccine composition be delivered in particulate form. For example, the vaccine composition can be administered using a particle acceleration device which fires nucleic acid-coated microparticles into target tissue, or transdermally delivers particulate nucleic acid compositions. In this regard, particle-mediated nucleic acid immunization has been shown to elicit both humoral and cytotoxic T lymphocyte immune responses following epidermal delivery of nanogram quantities of DNA. Pertmer et al. (1995) Vaccine 13:1427-1430. Particle-mediated delivery techniques have been compared to other types of nucleic acid inoculation, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:9519-9523. Such studies have investigated particle-mediated delivery of nucleic acid-based vaccines to both superficial skin and muscle tissue.

As described in detail herein above, particle-mediated methods for delivering nucleic acid preparations are known in the art. Thus, the polynucleotide vaccine composition can be coated onto core carrier particles using a variety of techniques known in the art. Carrier particles are selected from materials which have a suitable density in the range of particle sizes typically used for intracellular delivery from a particle acceleration device. The optimum carrier particle size will, of course, depend on the diameter of the target cells.

These methods can alternatively be modified by coadministration of additional or ancillary components to the subject. For example, a suitable adjuvant component can be added to the polynucleotide vaccine composition or administered along with the vaccine composition. In addition, a secondary vaccine composition can be administered, wherein the secondary composition can comprise a further nucleic acid vaccine, e.g., a polynucleotide encoding an additional influenza virus antigen derived or obtained from an influenza virus nucleoprotein (NP), neuraminidase (NA), hemagglutinin (HA), polymerase (PB1, PB2, PA), matrix (M1), or a non-structural (M2, NS1, NS2) gene product, or the secondary vaccine composition can comprise a conventional influenza vaccine such as a whole virus, split virus, or subunit influenza vaccine. The secondary vaccine composition can be combined with the polynucleotide vaccine composition to form a single composition, or the secondary vaccine composition can be administered separately to the same or to a different site, either concurrently, sequentially, or separated by a significant passage of time such as in a boosting step some days after the initial vaccine composition has been administered.

As above, the secondary vaccine composition and/or the adjuvant component can be administered by injection using either a conventional syringe, or using a particle-mediated delivery system as also described above. Injection will typically be either subcutaneously, epidermally, intradermally, intramucosally (e.g., nasally, rectally and/or vaginally), intraperitoneally, intravenously, orally or intramuscularly. Other modes of administration include topical, oral and pulmonary administration, suppositories, and transdermal applications. Dosage treatment may be a single dose schedule or a multiple dose schedule.

In another aspect, the method entails transfecting cells of the subject with a polynucleotide vaccine composition that includes one or more recombinant nucleic acid molecules having a sequence or sequences encoding one or more influenza virus M2 antigens (as described herein above). The transfection is carried out under conditions that permit expression of the M2 antigen in the subject, and the nucleic acid molecules are not present in a recombinant viral vector. Expression of the M2 antigen in situ is sufficient to elicit a protective immune response against an influenza virus. Transfection is effected using any of the above-described gene delivery techniques, with particle-mediated delivery being preferred. In addition, any of the secondary compositions, vaccine, adjuvant, or combinations thereof, can be used as described above.

### Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

Plasmid Construction

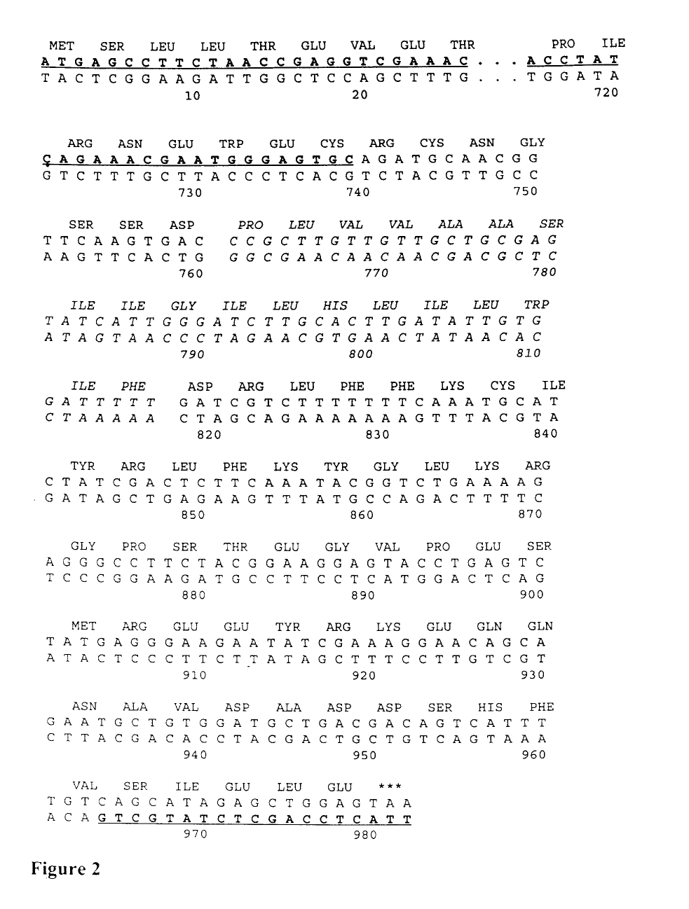

FIG. 2 shows the nucleotide sequence from RNA segment 7 (that encodes the M2 protein) of influenza virus strain A/Kagoshima/10/95 (H3N2). The A/Kagoshima sequence was used as a model to design polymerase chain reaction (PCR) primers to facilitate cloning of the mature M2 coding sequence from A/Sydney/5/97 (H3N2). The A/Kagoshima sequence was used for primer design since the sequence of RNA segment 7 of A/Sydney has not been determined. The high degree of conservation among M2 sequences was expected to facilitate the use of primers designed from a different viral strain.

Since M2 is translated from a spliced RNA, nucleotide positions 27 to 714 in the coding region of segment 7 RNA were spliced out. This is indicated by the gap between nucleotide positions 26 and 715 in the sequence shown in FIG. 2. The region of the coding sequence from segment 7 RNA that encodes the transmembrane portion of the M2 protein is shown in italics. The underlined sequences at the 5′ end (top strand) and 3′ end (bottom strand) represent the sequences from segment 7 RNA that were included in polymerase chain reaction (PCR) primers used to generate the complete M2 coding sequence. It should be noted that the 5′ PCR primer spans the RNA splice site shown in FIG. 2 to ensure that the intron was cleanly eliminated from M2 coding sequence clones derived by PCR. The 5′ and 3′ PCR primers used to generate the full-length M2 coding sequence clone were as follows:

| | |
|---|---|
| 5′ PCR Primer: | |
| 5′-CCC AAG CTT CCA CCA TGA GCC TTC TAA CCG AGG TCG AAA CAC | (SEQ ID NO:6) |
| CTA TCA GAA ACG AAT GGG AGT GC-3′ | |
| 3′ PCR Primer: | |
| 5′-CCC GGA TCC TTA CTC CAG CTC TAT GCT G-3′ | (SEQ ID NO:7). |

In addition to the M2 derived sequences indicated by underlining in FIG. 2, the 5′ PCR primer contains additional sequences at its 5′ end that include a recognition site for HindIII and a Kozak consensus sequence to facilitate mRNA translation initiation. Also, the 3′ PCR primer contains additional sequences at its 5′ end that includes a recognition sequence for BamHI.

Viral RNA was isolated from a sample of A/Sydney/5/97 (H3N2) that was grown in embryonated chicken eggs. The viral RNA isolation process used standard techniques known to those skilled in the art. RNA from this virus was used in a reverse transcriptase/ polymerase chain reaction (RT-PCR) using an RT-PCR kit obtained from Stratagene (La Jolla, Calif.). The RT reaction step was completed by adding 5.9 μl of RNase-free water to a reaction tube. To this tube was added 1.0 μl 10× MMLV-RT buffer and 1.0 μl dNTP mix from the kit. Also, 1 μl of A/Sydney/5/97 RNA and 0.6 μl (0.6 μg) of 5′ primer was added. The reaction was heated to 65° C. for 5 minutes to denature the RNA, after which 0.5 μl of reverse transcriptase from the kit was added. The reaction was incubated at 37° C. for 15 minutes to complete the reverse transcription step.

The PCR reaction step was completed by addition of the following components to a new reaction tube: 40 μl water; 5 μl 10× ultra HF buffer from the kit; 1.0 μl dNTP mix from the kit; 1.0 μl 5′ primer (1.0 μg); 1.0 μl 3′ primer (1.0 μg); 1 μl of the reverse transcriptase reaction mix from above; and 1 μl Turbo PFU polymerase from the kit. The PCR reaction was carried out using the following incubation scheme: 1 minute @ 95° C.; followed by 30 cycles of (30 sec @ 95° C., 30 sec @ 46° C., 3 min @ 68° C.), followed by 10 minutes @ 68 ° C. PCR products were electrophoresed on a 2% agarose gel revealing a single DNA band of the expected size of approximately 300 bp.

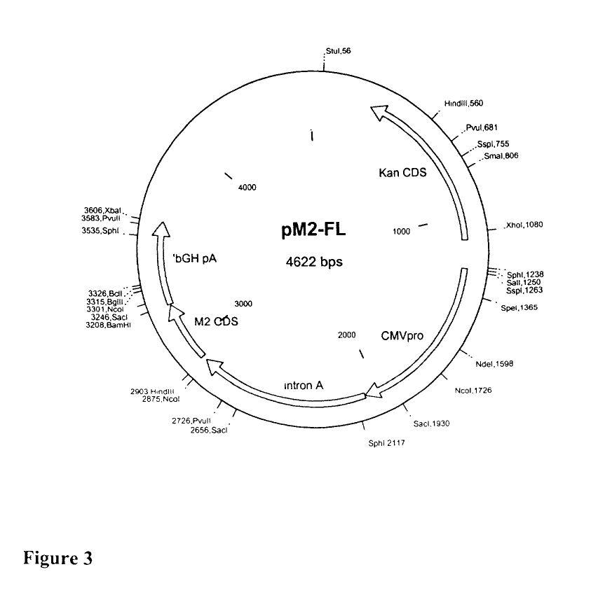

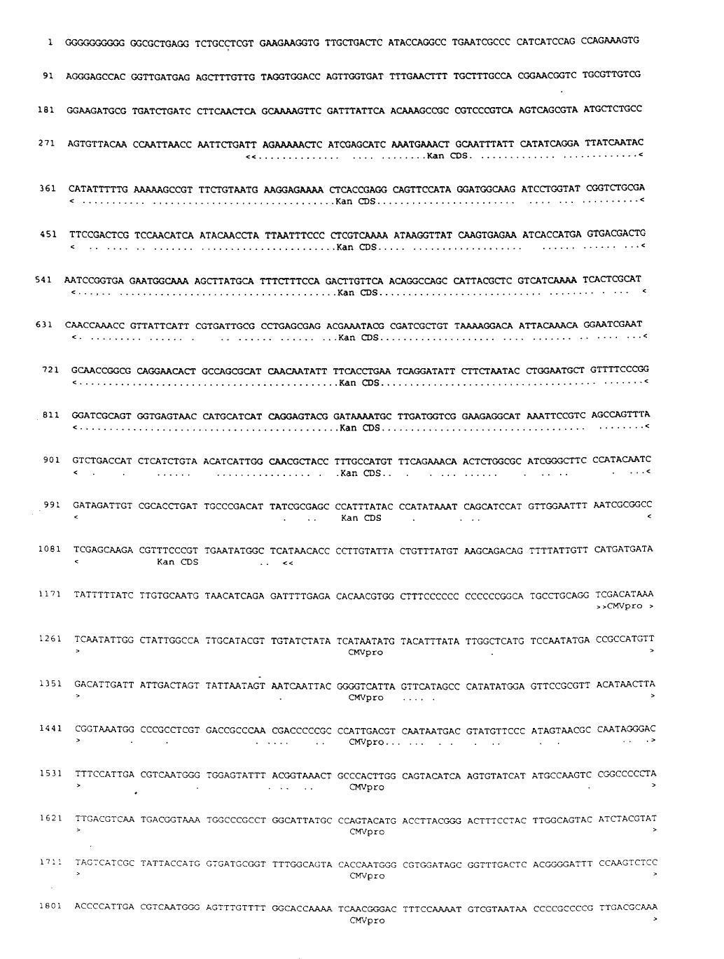

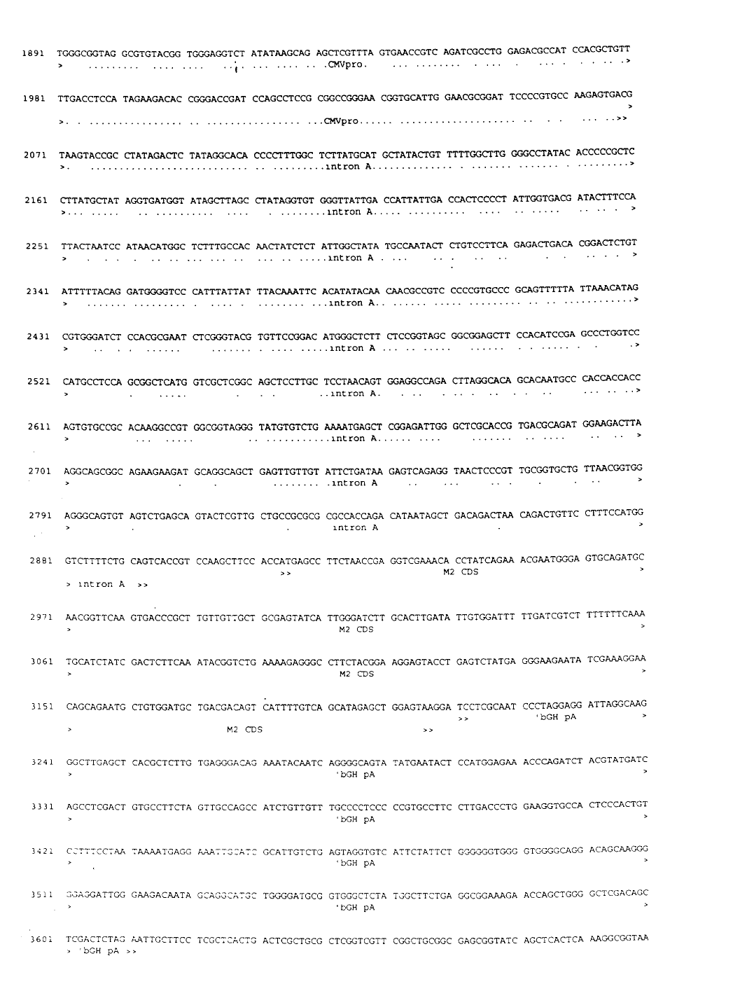

The approximately 300 bp band was isolated from the gel and digested with HindIII and BamHI in order to generate the necessary sticky ends for insertion into the pWRG7077 DNA vaccine expression vector (Schmaljohn et al. (1997) J. Virol. 71:9563-9569). The pWRG7077 DNA was digested partially with HindIII and completely with BamHI to facilitate insertion of the M2 coding insert. The requirement for a partial HindIII digestion of the vector was due to the presence of a second HindIII site in the Kanamycin resistance marker of this plasmid. The resulting M2 DNA vaccine vector was termed pM2-FL. The restriction map and functional map of this vector are shown in FIG. 3. The pM2-FL vector contains the immediate early promoter from human cytomegalovirus (hCMV) and its associated intron A sequence to drive transcription from the M2 coding sequence. This vector also includes a polyadenylation sequence from the bovine growth hormone gene.

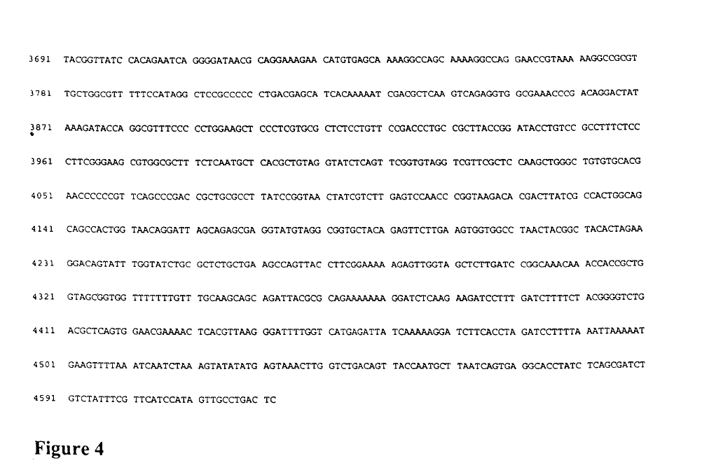

The annotated nucleotide sequence of the pM2-FL vector is shown in FIG. 4. The nucleotide sequence of the complete M2 coding sequence derived by the RT-PCR reaction was experimentally determined by standard sequencing methodologies known to those skilled in the art. The nucleotide sequence of the remaining part of the vector derived from plasmid pWRG7077 was deduced from the known sequence of pWRG7077.

It should be noted that the M2 coding sequence in plasmid pM2-FL differs from the known coding sequence of the M2 gene of A/Kagoshim/10/95 at 4 locations as follows:

A G:C base pair is found at nucleotide position #750 of the A/Kagoshima M2 sequence shown in FIG. 2. This position was determined to be an A:T pair in the A/Sydney-derived coding sequence inserted into pM2-FL. This nucleotide change results in a Glycine to Aspartic acid change at amino acid position 21 in the M2 protein. This change is consistent with the amino acid alignment shown in FIG. 1 in which position 21 is shown to be variable and that glycine and aspartic acid are the only observed amino acids at this position.

A T:A base pair is found at nucleotide position #815 in the A/Kagoshima M2 sequence shown in FIG. 2. This position was determined to be a C:G base pair in the A/Sydney-derived coding sequence inserted into pM2-FL. This nucleotide change results in a Phenylalanine to Leucine change at amino acid position #43 of the M2 protein. Of the 37 M2 sequences examined from the influenza virus sequence database, 7 viruses were shown to have a Phenylalanine residue at this location and 30 viruses were shown to have a Leucine residue at this location.

A T:A base pair is found at nucleotide position #857 in the A/Kagoshima M2 sequence shown in FIG. 2. This position was determined to be a C:G base pair in the A/Sydney-derived coding sequence inserted into pM2-FL. This nucleotide change results in a Tyrosine to Histidine change at amino acid position #57. Of the 37 M2 sequences examined from the influenza virus sequence database, 12 viruses were shown to have a Tyrosine at this location, 24 viruses were shown to have a Histidine at this location, and 1 virus was shown to have a glutamine at this location.

A T:A base pair is found at nucleotide position #862 in the A/Kagoshima M2 sequence shown in FIG. 2. This position was determined to be a C:G base pair in the A/Sydney-derived coding sequence inserted into pM2-FL. This nucleotide change does not result in any amino acid changes and is therefore a silent polymorphism.

In summary, the A/Sydney-derived M2 amino acid sequence differs from the A/Kagoshima M2 amino acid sequence at only three locations, but agrees with the majority of M2 sequences in the influenza virus sequence database at these three positions. This high degree of similarity is consistent with the high degree of conservation of M2 sequences among all type A influenza viruses.

### EXAMPLE 2

Induction of M2-Specific Antibody Responses in Mice

The following study was carried out in order to assess the ability to generate anti-M2 antibody responses using the nucleic acid immunization techniques of the present invention. 52 μg of the pM2-FL vector was added to 400 μl of 50 mM spermidine. 26 mg of micron-sized elemental gold particles (lot # 32-0, Degussa Corporation) was added to the reaction vessel. Finally, 400 μl of 10% calcium chloride was added while continuously agitating the mixture in order to precipitate the DNA onto the gold particles. DNA-laden gold particles were collected by centrifugation and washed three times with absolute ethanol then resuspended in 3.0 ml absolute ethanol. DNA-laden gold particles were loaded into 0.5 inch lengths of Tefzel tubing as previously described (see, e.g., U.S. Pat. Nos. 5,733,600 and 5,780,100, incorporated herein by reference).

Six mice received three consecutive particle-mediated DNA immunizations at four week intervals in which each immunization consisted of two particle-mediated deliveries of pM2-FL DNA coated gold particles. Each delivery contained 0.5 mg of gold and 1.0 μg of DNA for a total of 1 mg of gold and 2.0 μg of DNA per immunization. Gold/DNA deliveries were accomplished using a PowderJect XR-1 particle acceleration device (PowderJect Vaccines, Inc., Madison, Wis.) at a helium pressure of 400 p.s.i.

Blood samples were collected at the following time points: 4 weeks following the first immunization, 2 weeks following the second immunization (2 wk post boost), 4 weeks following the second immunization (4 wk post boost), and 2 weeks following the third immunization (2 wk post second boost). Sera were analyzed for M2-specific antibody responses using an ELISA assay in which 96-well plates were pre-coated with an M2 synthetic peptide consisting of the following sequence: HN₂-SLLTEVETPIRNEWECR-COOH (SEQ ID NO:8). ELISA plates were coated with the M2 peptide overnight at 4° C. using the peptide in phosphate buffered saline (PBS) at a concentration of 1 μg/ml. On the next day, plates were blocked with 5% nonfat dry milk in PBS for 1 hour at room temperature. Plates were then washed three times with wash buffer (10 mM Tris Buffered Saline, 0.1% Brij-35). Diluted serum samples were then added to the wells and the plates were incubated for 2 hours at room temperature. The plates were then washed three times with wash buffer. 100 μl of the secondary antibody was then added and plates were incubated for 1 hour at room temperature. The secondary antibody consisted of a goat anti-mouse IgG (H+L) biotin-labeled antibody (Southern Biotechnology) that was diluted 1:8000 in PBS/0.1% Tween-20. Plates were washed three times after which a streptavidin-horse radish peroxidase conjugate was added at a 1:8000 dilution. Following three washes, 100 μl of TMB substrate (Bio Rad) was added and color development was allowed to proceed for 30 minutes at room temperature. Color development was stopped by the addition of 1N H₂SO₄ and the plates were read of 450 nm.

Endpoint dilution titers were determined by identifying the highest dilution of serum that still yielded an absorbance value that was two times the background absorbance value obtained using a non-immune control sample. M2-specific antibody titers from individual animals, as well as the geometric mean titers are reported below in Table 1. 100% seroconversion was observed following the primary immunization and all animals developed an anamnestic or memory response following receipt of the second immunization.

TABLE 1

| | 4 wk post | 2 wk post | 4 wk post | 2 wk post |
|---|---|---|---|---|
| Mouse # | prime | boost 1 | boost 1 | boost 2 |
| 1 | 1200 | 10800 | 3600 | 10800 |
| 2 | 1200 | 32400 | 10800 | 32400 |
| 3 | 1200 | 10800 | 10800 | 97200 |
| 4 | 32400 | 97200 | 32400 | 291600 |
| 5 | 10800 | 32400 | 32400 | 32400 |
| 6 | 400 | 10800 | 3600 | 10800 |
| Geometric | 2496 | 22465 | 10800 | 38910 |
| mean | | | | |

### EXAMPLE 3:

Protective Immunity in Mice Immunized with pM2-FL DNA

The following study was carried out to demonstrate that nucleic acid immunization with an influenza virus M2 DNA sequence can be used to provide protective immunity in vaccinated subjects. More particularly, the study sought to determine whether vaccination in accordance with the present invention provides protection from death in a lethal influenza virus challenge model.

The mice immunized in Example 2 were anesthetized two weeks following the final immunization and were challenged intranasally with 1×10⁵ plaque forming units of mouse-adapted A/Aichi/2/68 (H3N2) which is 10 times the lethal dose for mice. Table 2, below, shows the percent survival of both the vaccinated (n=6) and non-immunized control (n=6) animals at various time points following challenge.

TABLE 2

| Day Post Challenge | pM2 DNA-Immunized | Control Group |
|---|---|---|
| 0 | 100% (6/6) | 100% (6/6) |
| 2 | 100% (6/6) | 100% (6/6) |
| 4 | 100% (6/6) | 100% (6/6) |
| 5 | 100% (6/6) | 100% (6/6) |
| 6 | 100% (6/6) | 83% (5/6) |
| 7 | 100% (6/6) | 0% (0/6) |
| 8 | 100% (6/6) | 0% (0/6) |
| 9 | 100% (6/6) | 0% (0/6) |
| 11 | 100% (6/6) | 0% (0/6) |
| 13 | 100% (6/6) | 0% (0/6) |
| 15 | 100% (6/6) | 0% (0/6) |
| 17 | 100% (6/6) | 0% (0/6) |

As can be seen in Table 2, while all control animals died by day 7, 100% survival was seen in the M2 DNA vaccine test group. Table 3, below, shows the percent weight loss in the vaccinated and challenge groups. The average weights of vaccinated and control (naive) mice are reported as percentage of starting weight at the time of challenge. All vaccinated mice became infected as evidenced by measurable weight loss following challenge, but these animals recovered and regained essentially all of the lost weight by the end of the experiment. In contrast, non-immunized control animals exhibited accelerated weight loss prior to death on days 6 and 7.

These observations are consistent with M2-specific antibodies being able to limit the spread of an infection and protect against disease, while being incapable of blocking the initial infection. These observations are novel and unique in that the M2-specific antibodies that provided protection in this case were elicited by induction of de novo production of M2 protein in vivo as a result of a nucleic acid immunization with an influenza virus M2 antigen sequence.

TABLE 3

| Day Post Challenge | pM2 DNA-Immunized | Control Group |
|---|---|---|
| 0 | 100.0% | 100.0% |
| 2 | 91.5% | 93.7% |
| 4 | 80.7% | 76.0% |
| 5 | 79.4% | 71.5% |
| 6 | 78.2% | 67.1% |
| 7 | 79.2% | DEAD |
| 8 | 81.1% | DEAD |
| 9 | 83.0% | DEAD |
| 11 | 87.3% | DEAD |
| 13 | 91.0% | DEAD |
| 15 | 95.0% | DEAD |
| 17 | 96.4% | DEAD |

### EXAMPLE 4

Induction of M2-Specific Antibody Response in Large Animal Model

The following study was carried out in order to assess the ability to generate M2-specific antibody responses in large animals using the nucleic acid immunization techniques of the present invention. The pM2-FL vector was coated onto gold particles as described in Example 2 with the exception that the DNA-to-gold ratio was increased from 2.0 μg DNA per mg of gold to 2.5 μg of DNA per mg gold. DNA-laden gold particles were formulated into 0.5-inch lengths of Tefzel tubing as previously described (see, e.g., U.S. Pat. Nos. 5,733,600 and 5,780,100, incorporated herein by reference). The final formulation consisted of 0.5 mg of gold and 1.25 μg of DNA per cartridge.

Nine 6-week old domestic Yorkshire/Landrace cross pigs were immunized three consecutive times at four week intervals in which each immunization consisted of six particle-mediated deliveries of pM2-FL DNA-coated gold particles. Each delivery contained 0.5 mg of gold and 1.25 μg of DNA for a total of 3 mg of gold and 7.5 μg of DNA per immunization. Vaccinations were administered bilaterally (3 shots per side) in the groin area without prior skin treatment of any kind. Gold/DNA deliveries were accomplished using a PowderJect XR-1 particle acceleration device (PowderJect Vaccines, Inc., Madison, Wis.) at a helium pressure of 500 p.s.i.

Blood samples were collected two weeks following the second and third immunizations. Sera were analyzed for M2-specific antibody responses using an ELISA assay in which 96-well plates were pre-coated with an M2 synthetic peptide consisting of the following sequence: NH₂-SLLTEVETPIRNEWECR-COOH. ELISA plates were coated with the M2 peptide overnight at 4° C. using the peptide in phosphate buffered saline (PBS) at a concentration of 1 μg/ml. On the next day, plates were blocked with 2% bovine serum albumin (BSA) in PBS for 1 hour at room temperature and were then washed three times with wash buffer (10 mM Tris-buffered saline, 0.1% Brij-35). Serum samples, diluted in 1% BSA/PBS/0.1% Tween-20, were added to the plates and incubated at room temperature for 2 hours. Plates were then washed three times with wash buffer. The detection antibody consisted of a goat anti-swine/horse radish peroxidase conjugate diluted 1:3200 in PBS/0.1% Tween-20. After addition of the diluted detection antibody, plates were incubated at room temperature for 60 minutes. Plates were again washed three times with was buffer and 100 μl of TMP substrate was added. After 20 minutes, color development was stopped by the addition of 1N H₂SO₄. Plates were read at 450 nm.

Endpoint dilution titers were determined by identifying the highest dilution of serum that still yielded an absorbance value that was two times the background absorbance value obtained using a non-immune control sample. M2-specific antibody titers from individual animals, as well as the geometric mean titers are reported below in Table 4. As can be seen from these results in Table 4, 100% seroconversion was observed following the final immunization. No detectable M2-specific antibody responses were observed in non-immunized control animals.

TABLE 4

| Pig # | Titer (Post Boost 1) | Titer (Post Boost 2) |
|---|---|---|
| 1 | 600 | 600 |
| 2 | 0 | 1800 |
| 3 | 200 | 200 |
| 4 | 200 | 1800 |
| 5 | 0 | 1800 |
| 6 | 0 | 600 |
| 7 | 0 | 1800 |
| 8 | 600 | 5400 |
| 9 | 0 | 600 |
| Geometric Mean | 13.4 | 1104 |

### EXAMPLE 5

Protective Immunity in Large Animals Immunized with pM2-FL DNA

The following study was carried out to demonstrate that nucleic acid immunization with an influenza virus M2 DNA sequence can be used to provide protection in large animals. More, particularly, the study sought to determine whether vaccination in accordance with the present invention provides for accelerated clearance of virus in a large animal following intranasal challenge.

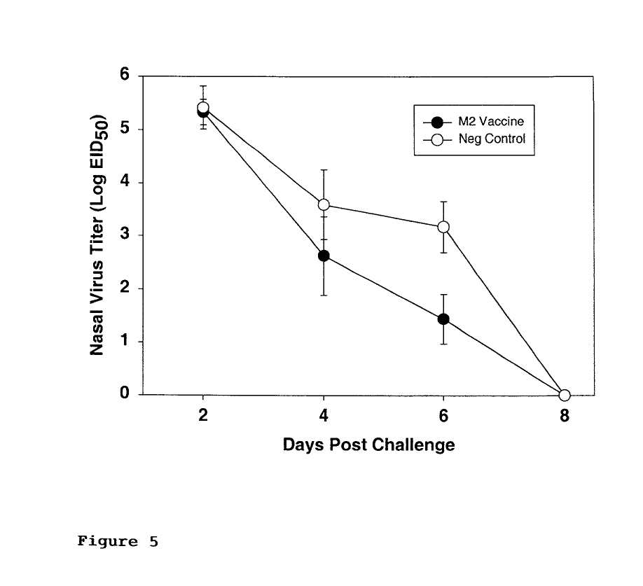

The pigs immunized in Example 4 were challenged approximately three weeks following the final immunization by intranasal instillation of 2×10⁶ egg infectious doses (EID₅₀) of live A/Swine/Minnesota/593/99 (1×10⁶ EID₅₀ per nostril). Nine negative control animals that were not vaccinated were similarly challenged. Nasal swab samples were collected on days 2, 4, 6, and 8 following challenge and the titer of virus in each sample was determined by titration in embryonated chicken eggs using standard procedures known to those skilled in the art of influenza virus growth. The graph depicted in FIG. 5 shows the geometric mean levels of virus in nasal swab samples in the vaccinated and negative control animals. As can be seen by reference to FIG. 5, the M2 DNA-vaccinated animals exhibited an accelerated clearance of virus in nasal swab samples relative to controls. This difference was most pronounced on day 6 in which there was a 53-fold reduction in the amount of live virus in vaccinated animals relative to non-vaccinated controls (P=0.021).

Accordingly, novel recombinant nucleic acid molecules, compositions comprising those molecules, and nucleic acid immunization techniques have been described. Although preferred embodiments of the subject invention have been described in some detail, it is understood that obvious variations can be made without departing from the spirit and the scope of the invention as defined by the appended claims.

What is claimed is:

1. A polynucleotide vaccine composition comprising a nucleic acid sequence that encodes an influenza virus M2 antigen, wherein said nucleic acid sequence is not present in a recombinant viral vector.

2. The composition of claim 1 wherein the nucleic acid sequence is present in a plasmid vector.

3. The composition of claim 1 wherein the nucleic acid sequence encodes an influenza virus M2 polypeptide.

4. The composition of claim 3 wherein the influenza virus M2 polypeptide comprises an amino acid sequence selected from the group consisting of SEQ ID NO:1, SEQ ID NO:2, SEQ ID NO:3, and hybrids or combinations thereof.

5. The composition of claim 1 further comprising a second nucleic acid sequence that encodes an influenza virus antigen derived or obtained from an influenza virus polypeptide selected from the group consisting of the nucleoprotein (NP), neuraminidase (NA), hemagglutinin (HA), polymerase (PB1, PB2, PA), matrix (M1), and non-structural (M2, NS1, NS2) gene products.

6. The composition of claim 1 further comprising an adjuvant component.

7. The composition of claim 6 wherein said adjuvant component is present in the composition in the form of a nucleic acid sequence.

8. The composition of claim 7 wherein said adjuvant component is a CpG sequence.

9. The composition of claim 7 wherein said adjuvant component is a further nucleic acid sequence that encodes a polypeptide adjuvant.

10. The composition of claim 6 wherein said adjuvant component is present in the composition in a form other than a nucleic acid sequence.

11. The composition of claim 10 wherein said adjuvant component is present in the composition in the form of a polypeptide.

12. The composition of claim 10 wherein said adjuvant component is present in the composition in the form of a lipid.

13. The composition of claim 10 wherein said adjuvant component is present in the composition in the form of a non-protein hormone or an analog thereof.

14. The composition of claim 10 wherein the adjuvant component is present in the composition in the form of a vitamin or an analog thereof.

15. The composition of claim 10 wherein the adjuvant component comprises monophosphoryl lipid A.

16. The composition of claim 10 wherein the adjuvant component comprises a saponin or a derivative thereof.

17. The composition of claim 16 wherein the adjuvant component comprises Quil-A.

18. The composition of claim 1 further comprising a pharmaceutically acceptable excipient or vehicle.

19. The composition of claim 1 wherein said composition is in particulate form.

20. The composition of claim 19 wherein the nucleic acid sequence is coated onto a core carrier particle.

21. The composition of claim 20 wherein the core carrier particle has an average diameter of about 0.1 to about 10 μm.

22. The composition of claim 20 wherein the core carrier particle comprises a metal.

23. The composition of claim 22 wherein the metal is gold.

24. A particle acceleration device suitable for use in a nucleic acid immunization technique, wherein said device is loaded with a particulate vaccine composition as defined in claim 19.

25. The composition of claim 1 further comprising a transfection facilitating agent.

26. A method for eliciting an immune response against an influenza virus in a subject, the method comprising administering the vaccine composition of claim 1 to the subject, whereby upon introduction to the subject, the nucleic acid sequence is expressed to provide the influenza virus M2 antigen in an amount sufficient to elicit said immune response.

27. The method of claim 26 wherein the vaccine composition is combined with a pharmaceutically acceptable vehicle and administered to the subject in the form of a liquid.

28. The method of claim 26 wherein the vaccine composition is administered directly into skin or muscle tissue.

29. The method of claim 26 wherein the vaccine composition is administered to mucosal tissue.

30. The method of claim 26 wherein the vaccine composition is administered by inhalation.

31. The method of claim 26 wherein the vaccine composition is administered topically.

32. The method of claim 26 wherein the vaccine composition is administered to the subject in particulate form.

33. The method of claim 26 wherein the nucleic acid sequence is coated onto a core carrier particle and administered to the subject using a particle-mediated delivery technique.

34. The method of claim 26 wherein the vaccine composition further comprises an adjuvant component.

35. The method of claim 26 further comprising the step of administering a second vaccine composition to the subject.

36. The method of claim 35 wherein the second vaccine composition is an anti-influenza vaccine selected from the group consisting of a whole virus vaccine, a subunit vaccine, a split vaccine, a nucleic acid vaccine, and any combination thereof.

37. The method of claim 35 wherein the second vaccine composition is administered to the subject in a boosting step.

38. The method of claim 35 wherein the vaccine composition of claim 1 and the second vaccine composition are administered to the same site in the subject.

39. The method of claim 35 wherein the vaccine composition of claim 1 and the second vaccine composition are administered concurrently.

40. The method of claim 35 wherein the vaccine composition of claim 1 and the second vaccine composition are combined to provide a single composition.

41. A method for using an influenza virus M2 antigen to induce an immune response in a subject, said method comprising:
(a) obtaining a nucleic acid sequence encoding the M2 antigen;
(b) providing an expression cassette by linking the nucleic acid sequence to regulatory sequences such that the nucleic acid sequence is operatively linked to control sequences that direct expression of the M2 antigen when introduced into tissue of the subject, wherein said expression cassette is not present in a recombinant viral vector; and
(c) administering the expression cassette to tissue of the subject.

42. The method of claim 41 wherein the expression cassette is present in a plasmid vector.

43. The method of claim 41 wherein the nucleic acid sequence encodes an influenza virus M2 polypeptide.

44. The method of claim 41 wherein the influenza virus M2 polypeptide comprises an amino acid sequence selected from the group consisting of SEQ ID NO:1, SEQ ID NO:2, SEQ ID NO:3, and hybrids or combinations thereof.

45. The method of claim 42 wherein the plasmid vector is administered directly into skin or muscle tissue of the subject.

46. The method of claim 45 wherein the plasmid vector is administered to the subject in particulate form.

47. The method of claim 45 wherein the plasmid vector is coated onto a core carrier particle and administered to the subject using a particle-mediated delivery technique.

48. The method of claim 41 wherein the subject is human.

49. A method of eliciting a protective immune response in a subject, said method comprising transfecting cells of the subject with a polynucleotide encoding an influenza virus M2 antigen, wherein said transfecting is carried out under conditions that permit expression of said antigen within the subject, said polynucleotide is not present in a recombinant viral vector, and said expression is sufficient to elicit a protective immune response against an influenza virus.

50. The method of claim 49 wherein the transfecting step is carried out in vivo using a particle-mediated transfection technique.


SEQUENCE LISTING (11 sequences)

SEQ ID NO: 1 - protein, 24 aa, Influenza A virus
MSLLTEVETPIRNEWECRCNGSSD

SEQ ID NO: 2 - protein, 24 aa, Influenza A virus
MSLLTEVETPIRNEWGCRCNDSSD

SEQ ID NO: 3 - protein, 24 aa, Influenza A virus
MSLLTEVETPIRNEWGCRCNGSSD

SEQ ID NO: 4 - DNA, 20 bp, Artificial Sequence
  Description of Artificial Sequence Synthetic Construct
tccatgacgttcctgatgct

SEQ ID NO: 5 - DNA, 20 bp, Artificial Sequence
  Description of Artificial Sequence Synthetic Construct
atcgactctcgagcgttctc

SEQ ID NO: 6 - DNA, 65 bp, Artificial Sequence
  Description of Artificial Sequence Synthetic Construct
cccaagcttccaccatgagccttctaaccgaggtcgaaacacctatcagaaacgaatgggagtgc

SEQ ID NO: 7 - DNA, 28 bp, Artificial Sequence
  Description of Artificial Sequence Synthetic Construct
cccggatccttactccagctctatgctg

SEQ ID NO: 8 - protein, 17 aa, Artificial Sequence
  Description of Artificial Sequence Synthetic Construct
SLLTEVETPIRNEWECR

SEQ ID NO: 9 - DNA, 294 bp, Influenza  A/Kagoshima/10/95(H3N2)
  CDS (1)..(291)
atgagccttctaaccgaggtcgaaacacctatcagaaacgaatgggagtgcagatgcaacggttcaagtgacccgcttgttgttgctgcgagtatcattgggatcttgcacttgatattgtggatttttgatcgtctttttttcaaatgcatctatcgactcttcaaatacggtctgaaaagagggccttctacggaaggagtacctgagtctatgagggaagaatatcgaaaggaacagcagaatgctgtggatgctgacgacagtcattttgtcagcatagagctggagtaa

SEQ ID NO: 10 - protein, 97 aa, Influenza  A/Kagoshima/10/95(H3N2)
MSLLTEVETPIRNEWECRCNGSSDPLVVAASIIGILHLILWIFDRLFFKCIYRLFKYGLKRGPSTEGVPESMREEYRKEQQNAVDADDSHFVSIELE

SEQ ID NO: 11 - DNA, 4622 bp, pM2-FL plasmid
ggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccggcatgcctgcaggtcgacataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccaagcttccaccatgagccttctaaccgaggtcgaaacacctatcagaaacgaatgggagtgcagatgcaacggttcaagtgacccgcttgttgttgctgcgagtatcattgggatcttgcacttgatattgtggatttttgatcgtctttttttcaaatgcatctatcgactcttcaaatacggtctgaaaagagggccttctacggaaggagtacctgagtctatgagggaagaatatcgaaaggaacagcagaatgctgtggatgctgacgacagtcattttgtcagcatagagctggagtaaggatcctcgcaatccctaggaggattaggcaagggcttgagctcacgctcttgtgagggacagaaatacaatcaggggcagtatatgaatactccatggagaaacccagatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgacagctcgactctagaattgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc